(19) United States
(12) Patent Application Publication
(10) Pub. No.: US 2024/0052122 A1
(43) Pub. Date: Feb. 15, 2024

(54) Novel Methods of Removing a Sacrificial Polymer in Polymer-Assisted Graphene Transfer; and Novel Bacillus Megaterium Strains, Related Compositions and Methods

(71) Applicant: University of North Carolina at Greensboro, Greensboro, NC (US)

(72) Inventors: Tetyana IGNATOVA, Whitsett, NC (US); Olubunmi O. AYODELE, Greensboro, NC (US); Swapna KALKAR, Greensboro, NC (US)

(21) Appl. No.: 18/379,157

(22) Filed: Oct. 11, 2023

Related U.S. Application Data
(63) Continuation-in-part of application No. PCT/US2023/017499, filed on Apr. 4, 2023.
(60) Provisional application No. 63/327,323, filed on Apr. 4, 2022.

(51) Int. Cl. C08J 7/12; C12N 1/20

(52) U.S. Cl. CPC C08J 7/12; C12N 1/205; C12R 2001/11

(57) ABSTRACT

Described herein are improved methods of removing a sacrificial polymer used in transfer of graphene from a formation substrate to a target substrate, novel graphene materials, novel Bacillus megaterium strains, and related compositions and methods of manufacture and methods of degrading polyvinyl alcohol.

### CROSS REFERENCE TO RELATED APPLICATIONS

This application is a continuation-in-part of Patent Cooperation Treaty Application No. PCT/US2023/017499 filed Apr. 4, 2023, which claims priority to U.S. Provisional Application No. 63/327,323 filed Apr. 4, 2022, both of which are incorporated herein by reference in their entirety.

### FIELD

Described herein are improved methods of removing a sacrificial polymer used in transfer of graphene from a formation substrate to a target substrate, novel graphene materials, novel Bacillus megaterium strains, and related compositions and methods of manufacture and methods of degrading polyvinyl alcohol, α-angelica lactone polymers, and other sacrificial polymers.

### BACKGROUND

Graphene is a monolayer thin-film having a planar honeycomb structure formed from chemical bonding of carbon atoms with a sp 2 hybrid orbital. Graphene has very high thermal conductivity and stability at the molecular level, high intrinsic carrier mobility (200,000 cm²/Vs), quantum electronic transport, tunable band gap, and high mechanical strength and elasticity. The advantageous properties of graphene make graphene an ideal material for high speed transistors, energy/thermal management, chemical/biological sensors and low power electronics. Graphene also provides an alternative to inorganic material, such as silicon which is fragile and size limited.

There are multiple methods of synthesizing graphene, including mechanical or chemical peeling, chemical vapor deposition on metal substrates, epitaxial synthesis, and organic synthesis. Chemical vapor deposition (CVD) is a preferred method because graphene of reasonable quality can be mass produced by this method inexpensively. CVD synthesizes graphene by adsorption, decomposition and segregation of a carbon-containing precursor on a formation substrate (e.g. a transition metal surface like copper foil, or conducting material) at an elevated temperature either at low or atmospheric pressure.

Graphene grown by CVD must be transferred from the conducting catalyst formation substrate onto a target substrate (e.g. SiO₂/Si or glass, an insulating material) in order for an electric current to flow through the graphene. This typically requires use of a sacrificial polymer (e.g. PMMA, PDMS, PC and PVA) as a support for the graphene while the formation substrate is removed (e.g. chemically etched) and the graphene is adhered to a target substrate. The sacrificial polymer is subsequently removed after transfer of the graphene onto the target substrate. This process is called polymer-assisted transfer of graphene. Current methods for removing the sacrificial polymer include dissolving the polymer with an organic solvent. However, polymer residues remain after solvent treatment and the solvent treatment itself is harsh and impacts the graphene material properties. The graphene produced by polymer-assisted transfer usually is highly cracked, strained, and/or doped. There remains a need to improve methods for a clean transfer of graphene from the formation substrate to the target substrate.

### SUMMARY OF THE INVENTION

The presently disclosed subject matter describes an isolated Bacillus megaterium strain having the deposit accession number B-6S:327 or the deposit accession number B-68328.

The presently disclosed subject matter describes an isolated Bacillus megaterium strain that can degrade polyvinyl alcohol (PVA, poly(vinyl) alcohol) without a bacterial symbiont. In some embodiments, the isolated Bacillus megaterium strain is obtained by culturing Bacillus megaterium in a medium comprising polyvinyl alcohol, peptone, yeast extract, and sodium chloride. In some embodiments, the isolated Bacillus megaterium strain is capable of degrading over 90% of a polyvinyl alcohol thin film from graphene without contaminating graphene. In some embodiments, the isolated Bacillus megaterium strain is capable of degrading polyvinyl alcohol (PVA, poly(vinyl) alcohol) without a bacterial symbiont and is averse to graphene.

The presently disclosed subject matter describes an isolated Bacillus megaterium strain that can degrade an α-angelica lactone polymer. In some embodiments, the isolated Bacillus megaterium strain is obtained by culturing Bacillus megaterium in a medium comprising an α-angelica lactone polymer having the structure:

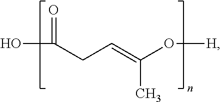

wherein n is a positive integer ranging from 1-100,000.

The presently disclosed subject matter describes a composition comprising an isolated Bacillus megaterium strain described herein and at least one of a carrier, diluent, surfactant, or adjuvant.

The presently disclosed subject matter describes a method of producing a Bacillus megaterium strain that degrades polyvinyl alcohol comprising culturing Bacillus megaterium in a medium comprising polyvinyl alcohol, peptone, yeast extract, and sodium chloride. In some embodiments, the Bacillus megaterium is cultured under an aerobic condition with constant shaking at 30° C. for 7 days. In some embodiments, the method further comprises transferring the culturing medium with the Bacillus megaterium into a second medium comprising peptone, yeast extract, and sodium chloride.

The presently disclosed subject matter describes a method of producing a Bacillus megaterium strain that degrades an α-angelica lactone polymer comprising: culturing Bacillus megaterium in a medium comprising an α-angelica lactone polymer having the structure:

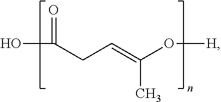

wherein n is a positive integer ranging from 1-100,000.

The presently disclosed subject matter describes a novel method for degrading polyvinyl alcohol comprising contacting the polyvinyl alcohol with a Bacillus megaterium strain described herein in the absence of a bacterial symbiont. In some embodiments, the polyvinyl alcohol is contacted with the isolated Bacillus megaterium strain having the deposit accession number B-68327 in the absence of bacterial symbiont PN19. The presently disclosed subject matter describes a novel method for degrading polyvinyl alcohol comprising contacting the polyvinyl alcohol with a composition comprising the Bacillus megaterium strain having the deposit accession number B-68327 and at least one of a carrier, diluent, surfactant, or adjuvant, wherein contacting the polyvinyl alcohol with the composition is in the absence of a bacterial symbiont.

The presently disclosed subject matter describes a method for degrading an α-angelica lactone polymer comprising contacting the α-angelica lactone polymer with a Bacillus megaterium strain described herein. In some embodiments, contacting the α-angelica lactone polymer with a Bacillus megaterium strain comprises contacting the α-angelica lactone polymer with a composition comprising the Bacillus megaterium strain having the deposit accession number B-68328 and at least one of a carrier, diluent, surfactant, or adjuvant.

The presently disclosed subject matter describes a method of removing a sacrificial polymer from graphene comprising providing a sacrificial polymer adhered to graphene, and contacting the sacrificial polymer with a Bacillus megaterium strain, wherein the Bacillus megaterium strain degrades the sacrificial polymer. In some embodiments, the Bacillus megaterium strain that degrades the sacrificial polymer was obtained by culture in a medium comprising the sacrificial polymer. In some embodiments, contacting the sacrificial polymer with a Bacillus megaterium strain comprises contacting the sacrificial polymer with a composition comprising the isolated Bacillus megaterium strain described herein and at least one of a carrier, diluent, surfactant, or adjuvant. In some embodiments, the sacrificial polymer is polyvinyl alcohol, polymethyl methacrylate, polydimethylsiloxane, polycarbonate, or an α-angelica lactone polymer having the structure:

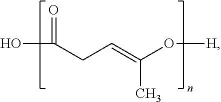

wherein n is a positive integer ranging from 1-100,000. In some embodiments, the sacrificial polymer is a film. In some embodiments, the graphene was grown by chemical vapor deposition on a formation substrate prior to adherence to the polyvinyl alcohol. In some embodiments, the formation substrate is a transition metal. In some embodiments, the formation substrate is copper foil. In some embodiments, the graphene is adhered to a target substrate. In some embodiments, the target substrate is an insulating material. In some embodiments, the target substrate is SiO₂/Si or glass. In some embodiments, the Bacillus megaterium strain degrades over 90% of the sacrificial polymer from the graphene. In some embodiments, the Bacillus megaterium strain is averse to graphene. In some embodiments, organic solvents are not used to remove the sacrificial polymer. In some embodiments, the Bacillus megaterium strain is the isolated Bacillus megaterium strain described herein. In some embodiments, the sacrificial polymer is polyvinyl alcohol and the Bacillus megaterium strain is the isolated Bacillus megaterium strain having the deposit accession B-68327, and wherein contacting the sacrificial polymer with the Bacillus megaterium strain occurs in the absence of a bacterial symbiont. In some embodiments, the isolated Bacillus megaterium strain having the deposit accession number B-68327 or the isolated Bacillus megaterium strain having the deposit accession number B-68328 degrades over 90% of the sacrificial polymer from the graphene. In some embodiments, the isolated Bacillus megaterium strain having the deposit accession number B-68327 degrades over 90% of the polyvinyl alcohol from the graphene. In some embodiments, the sacrificial polymer is polyvinyl alcohol and the Bacillus megaterium strain is the isolated Bacillus megaterium strain having the deposit accession [A], and wherein contacting the sacrificial polymer with the Bacillus megaterium strain occurs in the absence of a bacterial symbiont. In some embodiments, the sacrificial polymer is an α-angelica lactone polymer having the structure:

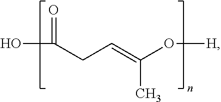

wherein n is a positive integer ranging from 1-100,000; and wherein, the isolated Bacillus megaterium strain has the deposit accession number B-68328. In some embodiments, the isolated Bacillus megaterium strain described herein does not contaminate the graphene. In some embodiments, the isolated Bacillus megaterium strain described herein is averse to graphene. In some embodiments, the polyvinyl alcohol is completely degraded and the graphene has improved optical and electronic properties as compared to graphene resulting from polyvinyl alcohol removal comprising a solvent treatment.

The presently disclosed subject matter describes graphene produced by the methods described herein. The presently disclosed subject matter describes novel graphene with improved optical and electronic properties.

### BRIEF DESCRIPTION OF THE DRAWINGS

The accompanying drawings, which are incorporated herein and form part of the specification, illustrate various embodiments of the present invention and, together with the description, further serve to explain the principles of the invention and to enable a person skilled in the pertinent art to make and use the invention. In the drawings, like reference numbers indicate identical or functionally similar elements.

FIG. 1 depicts an illustration of steps of a method of removing polyvinyl alcohol from graphene on SiO₂/Si (top) and an illustration of a cross-section of a sacrificial polymer adhered to graphene, which is adhered to a SiO₂/Si target substrate (bottom).

FIG. 2 is a line graph of optical density measurements over time for AB, BM, BM+PVA, AB+PVA, BM+PMMA, AB+PMMA, AB/BM+PVA, AB/BM+PMMA.

FIG. 3 is an SEM image of graphene after removal of the sacrificial polymer by a PVA-degrading bacteria in polymer-assisted transfer of graphene grown by CVD.

FIG. 4 is a Raman spectra of PVA (top) and a Raman spectra of graphene after removal of polyvinyl alcohol by contacting the polyvinyl alcohol with the isolated Bacillus megaterium strain described herein.

FIG. 5 is X-ray photoelectron spectra of C 1S for (1) graphene sample coated with PVA graphene (left), (2) graphene area after removal of polyvinyl alcohol by contacting the polyvinyl alcohol with the isolated Bacillus megaterium strain described herein (right red), and area outside of graphene (right navy). Inset shows normalized C 1S spectra; shift is a signature of sp² hybridized carbon in graphene.

FIG. 6 are images of growth of bacteria Bacillus megaterium on the surface of Si/SiO₂-graphene-PAL polymer on solid agar plate. Polymer thickness at Day 1 was about 1.0 mm. The inserts show progression of bacteria colony formation on the sample surface upon consumption of polymer.

FIG. 7 are before and after optical images that confirm the almost complete removal of PAL from the graphene surface. The optical image on the left is the before image, and the optical image on the right is after image after removing bacteria from the sample surface with water and ethanol (PAL polymer is not soluble in ethanol) on Day 10.

FIG. 8 show Raman characterization of transferred graphene over a large surface after degradation of the PAL sacrificial layer (2D band intensity): (left) a Raman image of graphene (2D band intensity), (top right) an SEM image of graphene, and (bottom right) a Raman spectra of graphene after removal of PAL by contacting the PAL with the isolated Bacillus megaterium strain described herein.

### DETAILED DESCRIPTION OF EMBODIMENTS

While the present invention may be embodied in many different forms, a number of illustrative embodiments are described herein with the understanding that the present disclosure is to be considered as providing examples of the principles of the invention and such examples are not intended to limit the invention to preferred embodiments described herein and/or illustrated herein. The claimed subject matter might also be embodied in other ways, to include different steps or elements similar to the ones described in this document, in conjunction with other present or future technologies. Moreover, although the term “step” may be used herein to connote different aspects of methods employed, the term should not be interpreted as implying any particular order among or between various steps herein disclosed unless and except when the order of individual steps is explicitly described.

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to elements throughout. Other details of the embodiments of the invention should be readily apparent to one skilled in the art from the drawings. Although the invention has been described based upon these preferred embodiments, it would be apparent to those skilled in the art that certain modifications, variations, and alternative constructions would be apparent, while remaining within the spirit and scope of the invention.

It is to be understood that all ranges described herein comprise all subranges therein. As illustration, the range “from 50 to 80” includes all possible ranges therein (e.g., 51-79, 52-78, 53-77, 54-76, 55-75, 60-70, etc.). Furthermore, all values within a given range include the endpoint of a range (e.g., the range 50-80 includes the ranges with endpoints such as 55-80, 50-75, etc.).

The presently disclosed subject matter is now described in more detail.

Technical problems are associated with a solvent treatment to remove polyvinyl alcohol from graphene in polymer-assisted transfer of graphene grown by CVD. Typically, the sacrificial polymer is dissolved by doping in bath of an organic solvent, such as acetone or chloroform, However, covalently bound polymer residues remain after solvent doping and can contaminate the graphene due to the strong dipole interactions between the polymer residue and the chemical groups on graphene. Polymer residue contamination causes a p-doping effect and impurity scattering. Moreover, the solvent treatment itself is harsh and impacts the graphene material properties. The graphene produced by polymer-assisted transfer usually is highly cracked, strained, and/or doped. Polymer residue contamination and solvent treatment interference with graphene's electronic and thermal properties are well recognized problems in the art. (Suk et al., 2013; Lin et al., 2019) The inventions described herein address these technical problems and the need for improved methods for a clean transfer of graphene from the formation substrate to the target substrate. In particular, described herein are improved methods of removing a sacrificial polymer used in transfer of graphene from a formation substrate to a target substrate, novel graphene materials, novel Bacillus megaterium strains, and related compositions and methods of manufacture and methods of degrading polyvinyl alcohol.

Bacteria-assisted removal of a sacrificial polymer from graphene is a novel approach in several aspects. First, a new isolated Bacillus megaterium strain that degrades PVA has been identified herein, and a new isolated Bacillus megaterium strain that degrades an α-angelica lactone polymer has been identified herein. Second, the standard solvent treatment step is removed altogether and replaced by an unconventional use of a polymer-degrading Bacillus megaterium strain. Moreover, because solvent doping can dissolve polymers relatively quickly (e.g. in less than 24 hours) and inexpensively, removal of this step in polymer-assisted transfer of graphene grown by CVD would not have been preferred or obvious.

A. Modified Bacillus megaterium, Related Compositions and Methods of Manufacture

Reported PVA-degrading microorganisms include gram-negative bacteria of the genus Pseudomonadaceae and the genus Sphingomonadaceae, some gram-positive bacteria, and eukaryotes such as fungi. Examples include: Pseudomonas O-3 (Suzuki et al. 1973); Pseudomonas putida VM15A cocultured with PQQ-producing Pseudomonas sp. VM 15C (Sakazawa et al. (1981); Pseudomonas vesicularis var. povalolyticus strain (Ishigaki et al. (1999)); Sphingomonas sp. SA3 cocultured with a growth factor producing SA2 strain (Kim et al. (2003)); fungal strains Penicillium daleae, Aspergillus flavus and Thalassospira povalilytica (Maiti et al. (2013) and Yuich et al. (2014)). See also Hoffmannn et al. (2003). Assessing biodegradability of plastics based on poly(viny1 alcohol) and protein wastes. J. Polym. Degrad. Stabil. 79:511-519; and Kawai et al., Appl Microbiol Biotechnol (2009) 84:227-237.

Patil et al. reported a strain of Bacillus subtilis (PVA-4) with 51% of PVA degradation in MSV-PVA medium supplemented with yeast extract and a strain of Pseudomonas aeruginosa (PVA-7) with 41% PVA degradation in MSV-PVA medium supplemented with yeast extract. (Patil 2014) PVA-4 and PVA-7 were derived from a mineral salt vitamin media (MSV medium (previously described by Suzuki et al. (1973): PVA, 5.0 g; (NH4)2SO4, 1.0 g; KH2PO4, 1.0 g; K2HPO4, 8.0 g; MgSO4.7H2O, 0.2 g; NaCl, 0.1 g; CaCl2.2H2O, 0.02 g; FeSO4, 0.01 g; Na2MoO4.2H2O, 0.5 mg; MnSO4, 0.5 mg; Inositol, 0.2 mg; p-amino benzoic acid, 0.2 mg; pyridoxine, 0.4 mg; thiamine, 2.0 μg; biotine, 2.0 μg; vitamin B, 120.5 μg; DW, 1000 ml; pH 7) enriched with soil/solid waste samples, industrial effluent samples, or polluted sea samples collected in India. (Patil et al. “Enrichment and isolation of microbial strains degrading bioplastic polyvinyl alcohol and time course study of their degradation potential.” African Journal of Biotechnology 14.27 (2015): 2216-2226.

Another reported PVA-degrading microorganism includes a strain of Bacillus megaterium as part of a mixed culture reported by Mori et al. The PVA-degrading mixed culture was produced by the method of an enrichment culture. Activated sludge in a textile factory was cultured in a PVA medium that contained PVA as the only carbon source. The PVA medium was composed of 0.1% PVA, 0.5% NH₄NO₃, 0.1% KH₂PO₄, 0.05% MgSO4, 7H₂O, 0.05% NaCl, and 0.02% yeast extract (pH 6.8). PVA-degrading microorgnisms were screened from the mixed culture. PVA-degrading Bacillus megaterium BX1 strain was isolated. However, thereafter, Bacillus megaterium BX1 strain showed poor growth and PVA degradation when cultured in PVA medium. Bacillus megaterium BX1 strain required the presence of a symbiotic partner, bacterial strain PN19, in order to degrade PVA. (Mori T, Sakimoto M, Kagi T, Sakai T (1996a) Isolation and characterization of a strain of Bacillus megaterium that degrades poly(vinyl alcohol). Biosci Biotech Biochem 60:330-332). Mori et al. also reported that a further mixed culture of Bacillus megaterium BX1 strain, its symbiotic partner—bacterial strain PN19, and a vinyl alcohol oligomer (OVA)-utilizing fungus strain WF9101 could degrade PVA. (Mori T, Sakimoto M, Kagi T, Sakai T (1996b) Degradation of vinyl alcohol oligomers by Geotrichum sp. WF9101. Biosci Biotech Biochem 60:1188-1190).

The presently disclosed subject matter describes a Bacillus megaterium strain with the following identifying characteristics:
(a) has polyvinyl alcohol-degrading activity in the absence of a bacterial symbiont,
(b) is capable of degrading over 90% of a polyvinyl alcohol thin film from graphene without contaminating graphene, and
(c) is averse to graphene.

“Averse to graphene” means preferentially migrates away from graphene, damaged by graphene (e.g. by membrane stress, or oxidative stress), or unable to grow or proliferate on graphene. A “bacterial symbiont” means bacterial participant in symbiotic relation with a modified Bacillus megaterium. In some embodiments, the isolated Bacillus megaterium strain is the isolated Bacillus megaterium strain having the deposit accession number B-68327

The presently disclosed subject matter describes an isolated Bacillus megaterium strain capable of degrading polyvinyl alcohol (PVA, poly(vinyl) alcohol) without a bacterial symbiont. In some embodiments, the isolated Bacillus megaterium strain is made by culturing Bacillus megaterium in a medium comprising polyvinyl alcohol, peptone, yeast extract, and sodium chloride. In some embodiments, the isolated Bacillus megaterium strain is the isolated Bacillus megaterium strain having the deposit accession number B-68327.

The presently disclosed subject matter describes a Bacillus megaterium strain with the following identifying characteristics:
(a) has α-angelica lactone polymer-degrading activity,
(b) is capable of degrading over 90% of an α-angelica lactone polymer thin film from graphene without contaminating graphene, and
(c) is averse to graphene.

“Averse to graphene” means preferentially migrates away from graphene, damaged by graphene (e.g. by membrane stress, or oxidative stress), or unable to grow or proliferate on graphene. In some embodiments, the isolated Bacillus megaterium strain is the isolated Bacillus megaterium strain having the deposit accession number B-68328.

The presently disclosed subject matter describes an isolated Bacillus megaterium strain capable of degrading α-angelica lactone polymer. In some embodiments, the isolated Bacillus megaterium strain is made by culturing Bacillus megaterium in a medium comprising an α-angelica lactone polymer having the structure:

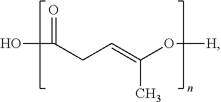

wherein n is a positive integer ranging from 1-100,000. In some embodiments, the isolated Bacillus megaterium strain is the isolated Bacillus megaterium strain having the deposit accession number B-68328.

The presently disclosed subject matter describes a composition comprising the isolated Bacillus megaterium strain described herein and at least one of a carrier, diluent, surfactant, or adjuvant. A “carrier” as defined herein is an inert, organic or inorganic material, with which the active ingredient is mixed or formulated to facilitate its application, or its storage, transport and/or handling. In some embodiments, the carrier is agar, or a liquid culture. In some embodiments, the surfactant is for the purpose of emulsification, dispersion, wetting, spreading, integration, disintegration control, stabilization of active ingredients, and improvement of fluidity.

The presently disclosed subject matter describes a method of producing a Bacillus megaterium strain that degrades polyvinyl alcohol comprising culturing Bacillus megaterium in a medium comprising with polyvinyl alcohol, peptone, yeast extract, and sodium chloride. In some embodiments, the Bacillus megaterium is cultured under an aerobic condition with constant shaking at 30° C. for 7 days. In some embodiments, the method further comprises transferring the culturing medium with the Bacillus megaterium into a second medium comprising peptone, yeast extract, and sodium chloride.

The presently disclosed subject matter describes a method of producing a Bacillus megaterium strain that degrades α-angelica lactone polymer comprising culturing Bacillus megaterium in a medium comprising with α-angelica lactone polymer. In some embodiments, the Bacillus megaterium is cultured under an aerobic condition with constant shaking at 30° C. for 7 days. In some embodiments, the method further comprises transferring the culturing medium with the Bacillus megaterium into a second medium comprising peptone, yeast extract, and sodium chloride.

B. Method of Degrading a Sacrificial Polymer

The presently disclosed subject matter describes a novel method for degrading polyvinyl alcohol comprising contacting the polyvinyl alcohol with the isolated Bacillus megaterium strain having the deposit accession number 13-68327 in the absence of a bacterial symbiont. In some embodiments, the polyvinyl alcohol is contacted with the isolated Bacillus megaterium strain described herein in the absence of bacterial symbiont PN19. The presently disclosed subject matter describes a novel method for degrading polyvinyl alcohol comprising contacting the polyvinyl alcohol with a composition comprising the isolated Bacillus megaterium strain having the deposit accession number B-68327 and at least one of a carrier, diluent, surfactant, or adjuvant, wherein contacting the polyvinyl alcohol with the composition is in the absence of a bacterial symbiont.

The presently disclosed subject matter describes a novel method for degrading α-angelica lactone polymer comprising contacting the α-angelica lactone polymer with the isolated Bacillus megaterium strain having the deposit accession number B-68328. The presently disclosed subject matter describes a novel method for degrading α-angelica lactone polymer comprising contacting the α-angelica lactone polymer with a composition comprising the isolated Bacillus megaterium strain having the deposit accession number B-68328 and at least one of a carrier, diluent, surfactant, or adjuvant.

“Degrading” as defined herein is degrading and/or consuming, and is a form of removal.

C. Method of Removing a Polymer from Graphene

The presently disclosed subject matter describes a method of removing a sacrificial polymer from graphene comprising providing a sacrificial polymer adhered to graphene, and contacting the sacrificial polymer with a microorganism, wherein the microorganism degrades the sacrificial polymer. In some embodiments, contacting the sacrificial polymer with a microorganism comprises contacting the sacrificial polymer with a composition comprising the isolated Bacillus megaterium strain described herein and at least one of a carrier, diluent, surfactant, or adjuvant. In some embodiments, contacting the sacrificial polymer with a microorganism comprises contacting the sacrificial polymer with a composition comprising the microorganism and at least one of a carrier, diluent, surfactant, or adjuvant. In some embodiments, the sacrificial polymer is polyvinyl alcohol, polymethyl methacrylate, polydimethylsiloxane, or polycarbonate. In some embodiments, the sacrificial polymer is a film. In some embodiments, the graphene was grown by chemical vapor deposition on a formation substrate prior to adherence to the sacrificial polymer. In some embodiments, the formation substrate is a transition metal. In some embodiments, the formation substrate is copper foil. In some embodiments, the graphene is adhered to a target substrate. In some embodiments, the target substrate is an insulating material. In some embodiments, the target substrate is SiO₂/Si or glass. In some embodiments, the over 90% of the sacrificial polymer is removed from graphene. In some embodiments, the microorganism is averse to graphene. In some embodiments, organic solvents are not used to remove the sacrificial polymer. In some embodiments, the method does not include a step to clean the graphene after removal of the sacrificial polymer, such as an ethanol cleaning step. In some embodiments, the microorganism is the isolated Bacillus megaterium strain having the deposit accession number 13-68327 in the absence of a bacterial symbiont. In some embodiments, the sacrificial polymer is polyvinyl alcohol and the microorganism is the isolated Bacillus megaterium strain having the deposit accession number B-68327 in the absence of a bacterial symbiont. In some embodiments, the isolated Bacillus megaterium strain described herein degrades over 90% of the polyvinyl alcohol from graphene. In some embodiments, the isolated Bacillus megaterium strain described herein does not contaminate the graphene. In some embodiments, the isolated Bacillus megaterium strain described herein is averse to graphene. In some embodiments, the polyvinyl alcohol is completely degraded and the graphene has improved optical and electronic properties as compared to graphene resulting from polyvinyl alcohol removal comprising a solvent treatment.

An exemplary protocol for bacteria-assisted removal of a polymer in polymer-assisted graphene transfer can include:
(a) growing or obtaining graphene on a formation substrate. Example formation substrates include copper, and other transition metals. Example methods for growing graphene include low-pressure chemical vapor deposition, atmospheric pressure chemical vapor deposition, plasma-enhanced chemical vapor deposition, Joule-heating chemical vapor deposition, and microwave chemical vapor deposition.
(b) forming a polymer support layer on the graphene side of graphene adhered to a formation substrate. The polymer support may be a thin film.
(c) removing the formation substrate. Example methods of removing the formation substrate include mechanical removal, plasma etching, chemical etching, and electrochemical removal.
(d) adhering the polymer-graphene substrate to a target substrate to form a polymer-graphene-target substrate. Example methods include hot press methods, electrochemical bubbling method, and physical placement method.
(e) utilizing a polymer-degrading microorganism to degrade/remove/consume the polymer. Example methods include adding the polymer-graphene-target substrate to a recess in agar (target substrate faced down) and adding a culture of polymer-degrading microorganism on the polymer of the polymer-graphene-target substrate. Example methods include permitting the polymer-graphene-target substrate to be treated 1, 2, 3, 4, or 5 days.
(f) obtaining clean graphene. Exemplary clean graphene has superior optical and electronic properties as compared to graphene obtained using a solvent treatment. Exemplary clean graphene obtained does not have polymer residues or bacterial contamination.

The presently disclosed subject matter describes graphene produced by the methods described herein. The presently disclosed subject matter describes clean graphene transfer. In some embodiments, the graphene is applied to a biosensor device, drug delivery compositions, bioimaging compositions, gene delivery compositions, phototherapy compositions, or tissue engineering compositions. In some embodiments, the graphene is applied to a flexible electronic device, or a transparent electronic device. In some embodiments, the graphene is applied to an electrode, a touch panel, an electroluminescent display, a backlight unit, an RFID tag, a solar cell module, an electronic paper, a TFT for a flat panel display, or a TFT array.

D. Deposit of Biological Material

Biological materials will be deposited with the Agricultural Research Service Culture Collection (NRRL):

| Identification | Accession Number | Date of Deposit |
|---|---|---|
| Bacillus megaterium | B-68327 | Oct. 31, 2023 |
| | Lab # Poly-TBS-PVA | |
| Bacillus megaterium | B-68328 | Oct. 31, 2023 |
| | Lab # Poly-TBS-PAL | |

Purified cultures of the microbial strains identified herein as an isolated Bacillus megaterium strain having the deposit accession number B-68327 and purified cultures of the microbial strains identified herein as an isolated Bacillus megaterium strain having the deposit accession number B-68328 will be deposited with the Agricultural Research Service Culture Collection (NRRL), 1815 North University Street, Peoria, Ill. 61604, U.S.A, in accordance with the Budapest Treaty for the purpose of patent procedure and the regulations thereunder (Budapest Treaty). The microbial strains have been deposited under conditions that ensure that access to the cultures will be available during the pendency of this patent application to one determined by the Commissioner of Patents and Trademarks to be entitled thereto under 37 C.F.R. § 1.14 and 35 U.S.C. § 122. The deposits represent substantially pure cultures of the deposited strains. The deposits are available as required by foreign patent laws in countries wherein counterparts of the subject application or its progeny are filed. However, it should be understood that the availability of a deposit does not constitute a license to practice the subject invention in derogation of patent rights granted by governmental action.

### Example 1: Isolated Bacillus megaterium Strain and Method of Manufacture

Materials and Methods:

Bacillus megaterium (4R6259 [Cornell 90]) and Aureobacterium barkeri (KDO37-2 [SCRF 322.0]) were obtained from ATCC. PVA (M_{w} 9,000-10,000, 80% hydrolyzed) was purchased from Sigma-Aldrich, United States and reconstituted to 5 wt % in distilled water while PMMA (Sigma-Aldrich, U.S.A; average M_{w} ˜120,000 by GPC) was reconstituted to 10 wt % in analytical grade acetone. The weight percent of PVA reconstituted in distilled water is not critical to producing PVA-degrading bacteria. Note that Bacillus megaterium (BM) and Aureobacterium barkeri (AB) in their unmodified form are not capable of degrading PVA.

Bacillus megaterium (BM) and Aureobacterium barkeri (AB) received in freeze-dried forms were grown in Luria-Bertani (LB) broth containing 10 g Peptone 140, 5 g Yeast Extract and 5 g Sodium Chloride at 30° C. for 24 h. The growth was monitored by measuring optical density (OD) at 600 nm using Fisherbrand™ accuSkan™ GO UV/Vis Microplate (96-plate well) Spectrophotometer. After 24 h, 10 μL of culture solution was transferred into 10 mL of LB broth media containing different concentrations of PVA and PMMA. The following bacteria and polymer combinations were prepared:
1) AB (Aureobacterium barkeri in LB broth),
2) BM (Bacillus megaterium in LB broth),
3) BM+PVA (mixed culture of Bacillus megaterium in LB broth and 20 mg of PVA),
4) AB+PVA (mixed culture of Aureobacterium barkeri in LB broth and 20 mg of PVA),
5) BM+PMMA (mixed culture of Bacillus megaterium in LB broth and 20 mg of PMMA),
6) AB+PMMA (mixed culture of Aureobacterium barkeri in LB broth and 20 mg of PMMA),
7) AB/BM+PVA (mixed culture of Aureobacterium barkeri and Bacillus megaterium in LB broth and 20 mg of PVA), and
8) AB/BM+PMMA (mixed culture of Aureobacterium barkeri and Bacillus megaterium in LB broth and 20 mg of PVA).

The above mixed cultures were incubated aerobically at 30° C. under constant shaking (100 rpm) for 7 days without replenishing the carbon source. The growth rate of bacteria was monitored periodically for 24 h and the Minimum Inhibitory Concentration (MIC) was obtained. Thereafter, 10 μL of the mixed culture solution is transferred into fresh LB broth and cultured at 30° C. under constant shaking (100 rpm) for 1 day.

Results:

The surviving bacteria were kept under 4° C. for further experiment.

The surviving bacteria from mixed culture #3 above is a Bacillus megaterium strain having the deposit accession number B-68327.

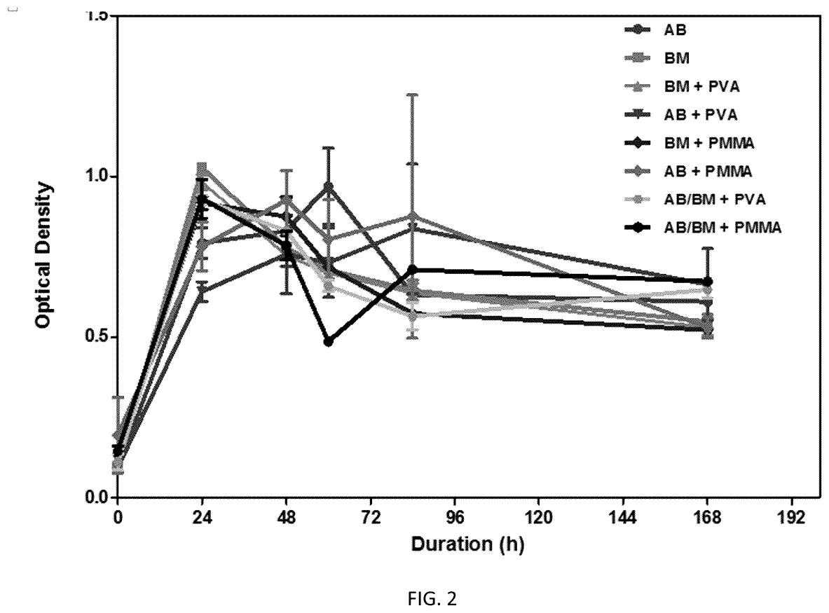

FIG. 2 is a line graph of optical density measurements over time for AB, BM, BM+PVA, AB+PVA, BM+PMMA, AB+PMMA, AB/BM+PVA, AB/BM+PMMA. Since we used single strain of BM and AB which have not been subjected to stress, their adaptability in a growth medium containing graphene sandwiched with SiO2/Si and polymer film (either PVA or PMMA) were investigated in agar plates. A noticeable increase in OD was observed for BM+PVA after 24 h incubation period and this is a testament to its ability to survive on PVA as a carbon source. In some instances, OD of AB+PVA, BM+PMMA, AB+PMMA only increased after 48 h of incubation. The bacteria initially formed cyst during acclimatization period; however BM+PVA was able to utilize PVA as carbon source and evolutionary modification of BM was achieved after 7 days of incubation without adding a fresh carbon source (PVA).

### Example 2: PVA Degradation by the Isolated Bacillus megaterium Strain in a Polymer-Assisted Graphene Transfer Protocol

Materials and Methods:

Graphene grown on Cu (formation substrate) by CVD was obtained under 2DCC Sample only grant #S0016 from Penn State. PVA (M_{w} 9,000-10,000, 80% hydrolyzed) was purchased from Sigma-Aldrich, United States and reconstituted to 5 wt % in distilled water. The weight percent of PVA reconstituted in distilled water is not critical to producing PVA-degrading bacteria.

In principle, graphene transfer with PVA thin film was done in accordance with a dry transfer technique. Graphene on Cu foil (formation substrate) was dip-coated with 5 wt % PVA solution and initially cured at 65° C. for 2 h and then at 90° C. for 3 h to form a thin PVA film on the graphene. Thereafter, hot-stamping of Cu-Gr-PVA was carried out intermittently to bring into contact graphene and the PVA thin film. Thereafter, the Gr-PVA film (PVA thin film fully adhered to graphene) was removed mechanically.

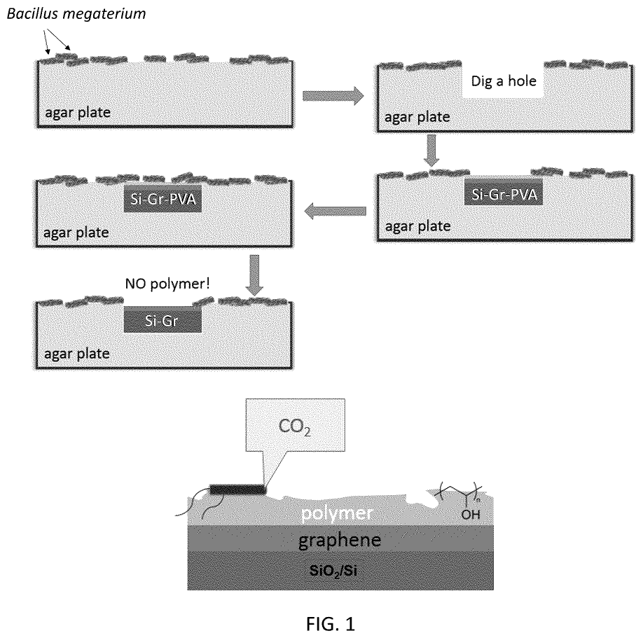

In order to improve the contact between Graphene-PVA film and SiO₂/Si (target substrate), the surface of an ozone-treated SiO₂/Si was made hydrophilic using isoprophyl alcohol (IPA) (HPLC grade) solvent treatment. The Gr-PVA film was then transferred onto or deposited on the SiO₂/Si target substrate, with graphene sandwiched and in contact with both the PVA film and SiO₂/Si target substrate. FIG. 1 (bottom illustration) is an illustration of an exemplary cross-section of a sacrificial polymer adhered to graphene, which is adhered to a target substrate, such as SiO₂/Si.

Thereafter, in order to enhance the contact of Gr-PVA thin film with the surface of SiO₂/Si thereof, a drop of distilled H₂O onto the surface of PVA-Gr-SiO₂/Si film was done, hot stamped for 30 sec and dried by bubbling N₂ air.

FIG. 1 (top illustration) depicts an illustration of steps of a method of removing polyvinyl alcohol from graphene on SiO₂/Si. Sterile LB agar was prepared and sterilized in an autoclave and it was left cool to 60° C. before being poured into agar plates in a sterile inoculating chamber. Thereafter, the SiO₂/Si-Gr-PVA substrate was placed at the center of agar plate and digged inside the agar so that the surface of the substrate can be on the same level with the agar. 10 μL of culture containing surviving bacteria from mixed culture #3 (Bacillus megaterium strain having the deposit accession number B-68327) was introduced and streaked aseptically on the solidified agar and then transferred to an incubator set at 30° C. No bacterial symbiont was included in the culture. No ethanol cleaning step of the graphene is required.

After the third day, Raman spectroscopy and X-ray photoelectron spectroscopy was performed, and an SEM image was taken.

Results:

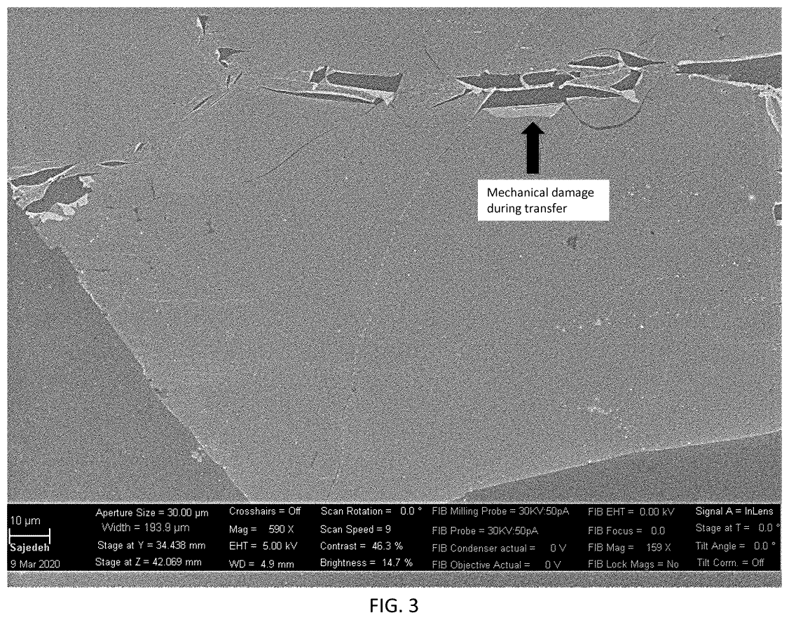

After the 3rd day, PVA thin film covering was degraded and removed while the surface covered with graphene had no bacterial contamination based on scanning electron micrograph, FIG. 3 is an SEM image of graphene after removal of the sacrificial polymer by the PVA-degrading bacteria in polymer-assisted transfer of graphene grown by CVD.

Bacteria-assisted removal of polyvinyl alcohol from graphene in polymer-assisted graphene transfer resulted in a superior clean transfer with no contamination from either polymer or bacterial cells. Once the polymer was degraded or consumed by the bacteria from mixed culture #3 (Bacillus megaterium strain having the deposit accession number B-68327), the bacteria was averse to the graphene and migrated to the agar plate to continue their proliferation.

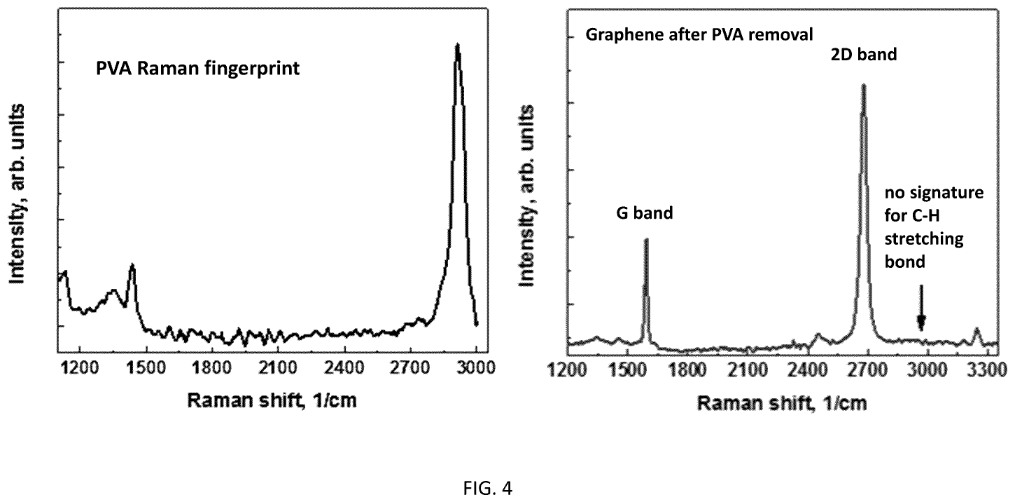

FIG. 4 is a Raman spectra of PVA (top) and a Raman spectra of graphene after removal of polyvinyl alcohol by contacting the polyvinyl alcohol with the isolated Bacillus megaterium strain described herein. A PVA fingerprint was not detected by Raman spectroscopy.

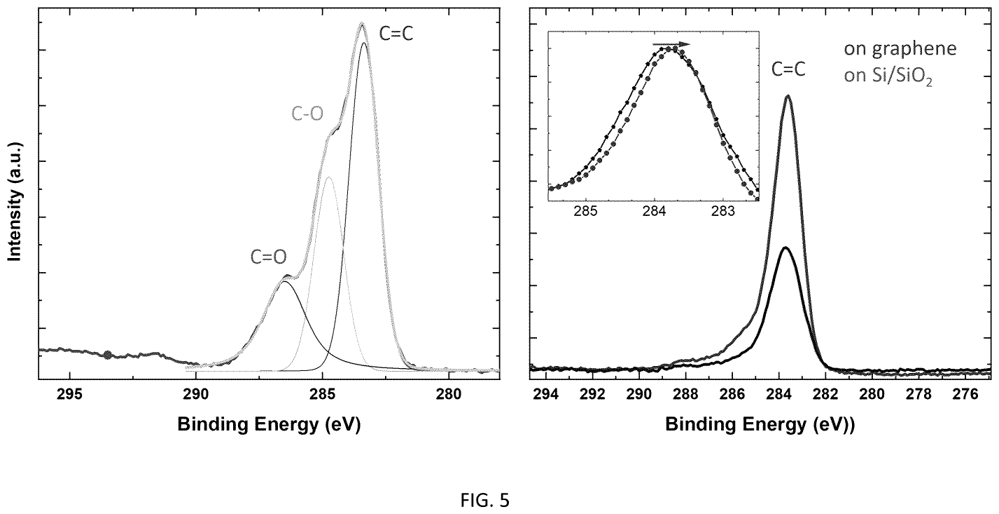

FIG. 5 is X-ray photoelectron spectra of C 1S for (1) graphene sample coated with PVA graphene (left), (2) graphene area after removal of polyvinyl alcohol by contacting the polyvinyl alcohol with the isolated Bacillus megaterium strain described herein (right, red), and area outside of graphene (right navy). Inset shows normalized C 1S spectra; shift is a signature of sp² hybridized carbon in graphene. The photoelectron spectra measures the amount of polymer left on the substrate. Over 90% of the PVA of the SiO₂/Si-Gr-PVA substrate was removed after three days which is higher than the solvent removal method.

### Prophetic Example 3: PMMA Degradation by the Isolated Bacillus megaterium Strain in a Polymer-Assisted Graphene Transfer Protocol

Graphene grown on Cu by chemical vapor deposition (CVD) was obtained as a gift from PennState. PMMA (Sigma-Aldrich, U.S.A; average M_{w} ˜950,000 by GPC) was reconstituted to 10 wt % in analytical grade acetone. The weight percent of PMMA reconstituted in distilled water is not critical to producing PVA-degrading bacteria.

Conventional PMMA transfer was conducted using a procedure developed by Hallam et al. (2014)

The protocol for PMMA removal by the surviving bacteria from mixed culture #5 above is similar to the polymer removal steps in Example 2. Sterile LB agar will be prepared and sterilized in an autoclave and left cool to 60° C. before being poured into agar plates in a sterile inoculating chamber. Thereafter, the SiO₂/Si-Gr-PMMA substrate will be placed at the center of agar plate and digged inside the agar so that the surface of the substrate can be on the same level with the agar. 10 μL of culture containing surviving bacteria from mixed culture #5 above will be introduced and streaked aseptically on the solidified agar and then transferred to an incubator set at 30° C. No bacterial symbiont is included in the culture. No ethanol cleaning step of the graphene is required.

### Prophetic Example 4: Improved Graphene Properties

Due to the clean transfer of graphene possible by the isolated Bacillus megaterium strain and bacteria-assisted removal of polyvinyl alcohol from graphene in polymer-assisted graphene transfer described herein, the resulting graphene is not contaminated by PVA or altered by a solvent treatment. Accordingly, it is expected that the resulting graphene will advantageously have significantly improved optical and electronic properties.

### Example 5

Isolated Bacillus megaterium Strain and Method of Manufacture

Materials and Methods:

1. Bacillus megaterium (4R6259 [Cornell 90]) was obtained from ATCC. Note that Bacillus megaterium (BM) in their unmodified form are not capable of degrading PAL. An α-angelica lactone polymer (PAL) having the structure:

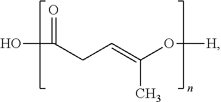

wherein n is a positive integer ranging from 1-100,000 was produced by polymerization of PAL following previously published procedures:
J. Xin, S. Zhang, D. Yan, O. Ayodele, X. Lu, and J. Wang, “Formation of C—C bonds for the production of bio-alkanes under mild conditions,” Green Chemistry, vol. 16, no. 7, pp. 3589-3595, 2014, doi: 10.1039/c4gc00501e.
C. G. S. Lima, J. L. Monteiro, T. de Melo Lima, M. Weber Paixão, and A. G. Corrêa, “Angelica Lactones: From Biomass-Derived Platform Chemicals to Value-Added Products,” ChemSusChem, vol. 11, no. 1. Wiley-VCH Verlag, pp. 25-47, Jan. 10, 2018. doi: 10.1002/cssc.201701469.

T. Chen et al., “Degradable polymers from ring-opening polymerization of α-angelica lactone, a five-membered unsaturated lactone,” Polym Chem, vol. 2, no. 5, pp. 1190-1194, 2011, doi: 10.1039/c1py00067e.

Bacillus megaterium (BM) was received in freeze-dried forms were grown in Luria-Bertani (LB) broth containing 10 g Peptone 140, 5 g Yeast Extract and 5 g Sodium Chloride at 30° C. for 24 h. The growth was monitored by measuring optical density (OD) at 600 nm using Fisherbrand™ accuSkan™ GO UV/Vis Microplate (96-plate well) Spectrophotometer. After 24 h, 10 μL of culture solution was transferred into 10 mL of LB broth media with 20 mg of PAL. The mixed cultures was incubated aerobically at 30° C. under constant shaking (100 rpm) for 7 days without replenishing the carbon source. The growth rate of bacteria was monitored periodically for 24 h and the Minimum Inhibitory Concentration (MIC) was obtained. Thereafter, 10 μL of the mixed culture solution was transferred into fresh LB broth and cultured at 30° C. under constant shaking (100 rpm) for 1 day.

Results:

The surviving bacteria were kept under 4° C. for further experiment. The surviving bacteria from the mixed culture with PAL is a Bacillus megaterium strain having the deposit accession number B-68328.

PAL Degradation by the Isolated Bacillus megaterium Strain in a Polymer-Assisted Graphene Transfer Protocol in the Absence of an Organic Solvent

Materials and Methods:

Graphene grown on Cu (formation substrate) by CVD was obtained under 2DCC Sample only grant #S0016 from Penn State. PAL was produced as described above.

In principle, graphene transfer with PAL thin film was done in accordance with a dry transfer technique. Graphene on Cu foil (formation substrate) was dip-coated with 5 wt % PAL solution and initially cured at 65° C. for 2 h and then at 90° C. for 3 h to form a thin PAL film on the graphene. Thereafter, hot-stamping of Cu-Gr-PAL was carried out intermittently to bring into contact graphene and the PAL thin film. Thereafter, the Gr-PAL film (PAL thin film fully adhered to graphene) was removed mechanically.

In order to improve the contact between Graphene-PAL film and SiO₂/Si (target substrate), the surface of an ozone-treated SiO₂/Si was made hydrophilic using isoprophyl alcohol (IPA) (HPLC grade) solvent treatment. The Gr-PAL film was then transferred onto or deposited on the SiO₂/Si target substrate, with graphene sandwiched and in contact with both the PAL film and SiO₂/Si target substrate.

Thereafter, in order to enhance the contact of Gr-PAL thin film with the surface of SiO₂/Si thereof, a drop of distilled H₂O onto the surface of PAL-Gr-SiO₂/Si film was done, hot stamped for 30 sec and dried by bubbling N₂ air.

Sterile LB agar was prepared and sterilized in an autoclave and it was left cool to 60° C. before being poured into agar plates in a sterile inoculating chamber. Thereafter, the SiO₂/Si-Gr-PAL substrate was placed at the center of agar plate and digged inside the agar so that the surface of the substrate can be on the same level with the agar. 10 μL of culture containing surviving bacteria from mixed culture with PAL (Bacillus megaterium strain having the deposit accession number B-68328) was introduced and streaked aseptically on the solidified agar and then transferred to an incubator set at 30° C. No ethanol cleaning step of the graphene is required.

After the third day, Raman spectroscopy and X-ray photoelectron spectroscopy was performed, and an SEM image was taken.

Results:

On day 10, the PAL thin film covering was degraded and removed while the surface covered with graphene had no bacterial contamination. PAL was degraded by the bacteria from mixed culture with PAL (Bacillus megaterium strain having the deposit accession number B-68328) in the absence of organic solvent (Note: PAL is soluble in methylene chloride, acetone, ethyl acetate and methanol, PAL is not soluble in water)

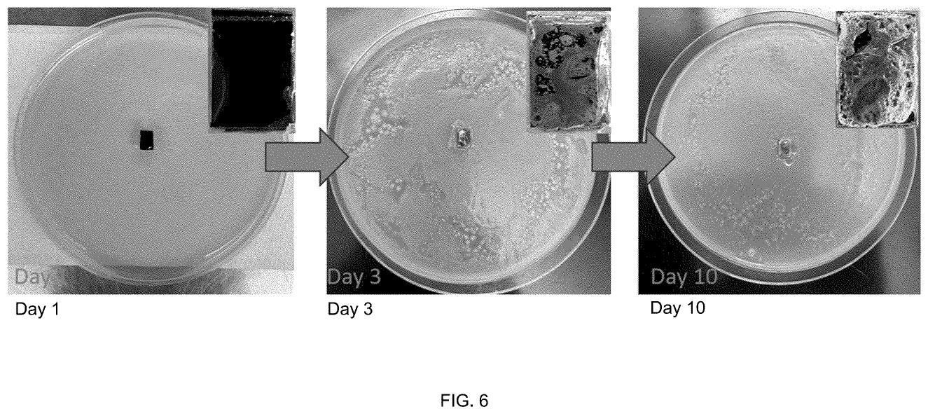

FIG. 6 are images of growth of bacteria Bacillus megaterium on the surface of Si/SiO2-graphene-PAL polymer on solid agar plate over time (Day 1, Day 3, Day 10). Polymer thickness at Day 1 was about 1.0 mm. The inserts show progression of bacteria colony formation on the sample surface upon consumption of polymer.

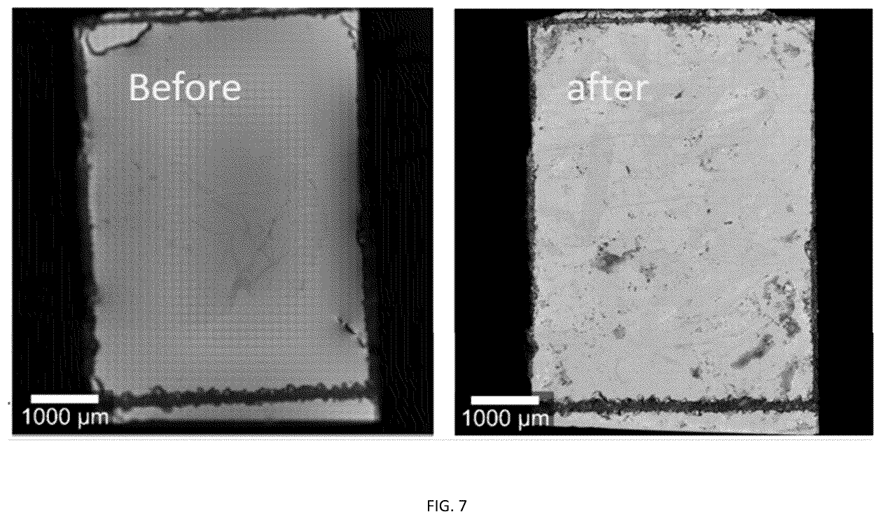

FIG. 7 are before and after optical images that confirm the almost complete removal of PAL from the graphene surface. The optical image on the left is the before image of transferred graphene (PAL covered, thickness 1 mm) on the target substrate SiO2/Si before introduction of the microorganism, and the optical image on the right is image on the 10^{th} day of exposure to the Bacillus megaterium strain having the deposit accession number B-68328 and after removing bacteria from the sample surface with DI water from the graphene surface.

The sample was characterized with scanning electron microscopy and Raman spectroscopy.

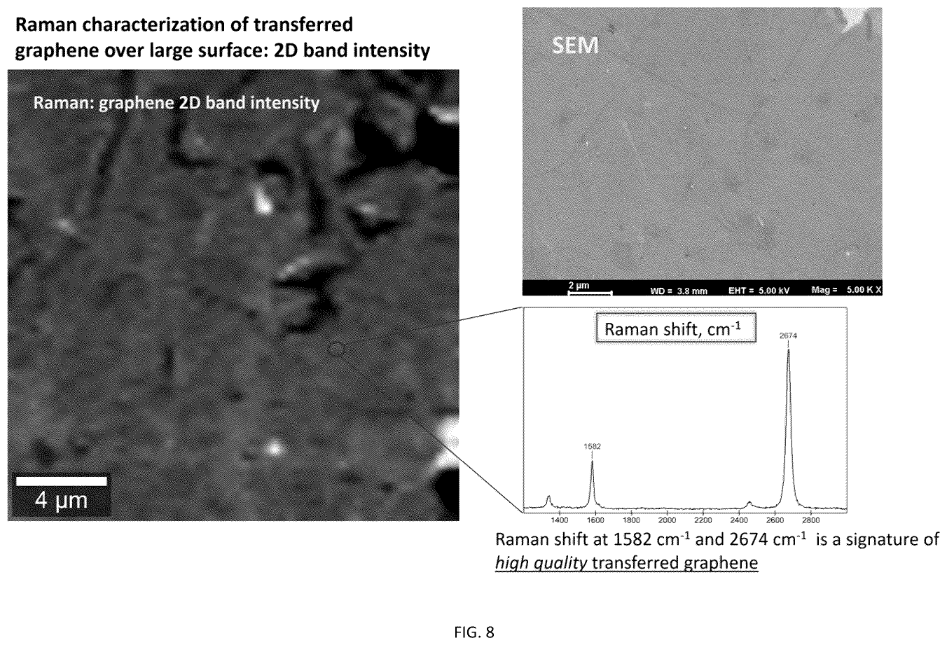

FIG. 8 show Raman characterization of transferred graphene over a large surface after degradation of the PAL sacrificial layer (2D band intensity): (left) a Raman image of graphene (2D band intensity), (top right) an SEM image of graphene, and (bottom right) a Raman spectra of graphene after removal of PAL by contacting the PAL with the isolated Bacillus megaterium strain described herein.

Bacteria-assisted removal of PAL from graphene in polymer-assisted graphene transfer resulted in a superior clean transfer with no contamination from either polymer or bacterial cells. Once the polymer was degraded or consumed by the bacteria from mixed culture with PAL (Bacillus megaterium strain having the deposit accession number B-68328), the bacteria was averse to the graphene and migrated to the agar plate to continue their proliferation. The PAL is completely degraded and the graphene optical and electronic properties are preserved.

### REFERENCES

Chikahiro Sakazawa, Masayuki Shimao, Yoshifumi Taniguchi, and Nobuo Kato (1981). Symbiotic Utilization of Polyvinyl Alcohol by Mixed Cultures. Applied and Environmental Microbiology, 41, 261-267.
Hallam, T., Berner, N. C., Yim, C., & Duesberg, G. S. (2014). Strain, Bubbles, Dirt, and Folds: A Study of Graphene Polymer-Assisted Transfer. Advanced Materials Interfaces, 1(6), 1400115. doi:10.1002/admi.201400115
Hoffmann, J., R̆eznic̆ková, I., Kozáková, J., Růz̆ic̆ka, J., Alexy, P., Balos̆, D., & Precnerova, L. (2003). Assessing biodegradability of plastics based on poly(vinyl alcohol) and protein wastes. Polymer Degradation and Stability, 79(3), 511-519. doi:10.1016/s0141-3910(02)00367-1
Kawai, F. (1995). Breakdown of plastics and polymers by microorganisms. Advances in Biochemical Engineering/Biotechnology, 151-194. doi:10.1007/bfb0102319
Kim, B. C., Sohn, C. K., Lim, S. K., Lee, J. W., & Park, W. (2003). Degradation of polyvinyl alcohol by Sphingomonas sp. SA3 and its symbiote. Journal of Industrial Microbiology & Biotechnology, 30(1), 70-74. doi: 10.1007/s10295-002-0010-4
Lin, L., Zhang, J., Su, H., Li, J., Sun, L., Wang, Z., . . . Liu, Z. (2019). Towards super-clean graphene. Nature Communications, 10(1). doi:10.1038/s41467-019-09565-4
Maiti, S., Ray, D., Mitra, D., & Mukhopadhyay, A. (2013). Isolation and characterisation of starch/polyvinyl alcohol degrading fungi from aerobic compost environment. International Biodeterioration & Biodegradation, 82, 9-12. doi: 10.1016/j.ibiod.2013.01.018
Mori, T., Sakimoto, M., Kagi, T., & Sakai, T. (1996). Isolation and Characterization of a Strain of Bacillus megaterium That Degrades Poly(vinyl alcohol). Bioscience, Biotechnology, and Biochemistry, 60(2), 330-332. doi:10.1271/bbb.60.330
Nogi, Y., Yoshizumi, M., & Miyazaki, M. (2014). Thalassospira povalilytica sp. nov., a polyvinyl-alcohol-degrading marine bacterium. International Journal of Systematic and Evolutionary Microbiology, 64(Pt 4), 1149-1153. doi:10.1099/ijs.0.058321-0
Rajashree, P., & U, S. B. (2015). Enrichment and isolation of microbial strains degrading bioplastic polyvinyl alcohol and time course study of their degradation potential. African Journal of Biotechnology, 14(27), 2216-2226. doi:10.5897/ajb2011.3980
Suk, J. W., Lee, W. H., Lee, J., Chou, H., Piner, R. D., Hao, Y., Akinwande, D., Ruoff, R. S. (2013). Enhancement of the Electrical Properties of Graphene Grown by Chemical Vapor Deposition via Controlling the Effects of Polymer Residue. Nano Letters, 13(4), 1462-1467. doi:10.1021/n1304420b
Suzuki, T., Ichihara, Y., Yamada, M., & Tonomura, K. (1973). Some Characteristics of Pseudomonas0-3 which Utilizes Polyvinyl Alcohol. Agricultural and Biological Chemistry, 37(4), 747-756. doi:10.1080/00021369.1973.10860756
Tatsuma, M., Michio, S., Takashi, K., & Takuo, S. (1996). Degradation of Vinyl Alcohol Oligomers by Geotrichumsp. WF9101. Bioscience, Biotechnology, and Biochemistry, 60(7), 1188-1190. doi:10.1271/bbb.60.1188

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

1. An isolated Bacillus megaterium strain having the deposit accession number B-68327 or the deposit accession number B-68328.

2. A composition comprising the isolated Bacillus megaterium strain of claim 1 and at least one of a carrier, diluent, surfactant, or adjuvant.

3. A method of producing a Bacillus megaterium strain that degrades polyvinyl alcohol comprising:
culturing Bacillus megaterium in a medium comprising polyvinyl alcohol, peptone, yeast extract, and sodium chloride.

4. The method of claim 3, wherein Bacillus megaterium is cultured under an aerobic condition with constant shaking at 30° C. for 7 days.

5. The method of claim 3, further comprising transferring the culturing medium with the Bacillus megaterium into a second medium comprising peptone, yeast extract, and sodium chloride.

6. A method for degrading polyvinyl alcohol comprising: contacting the polyvinyl alcohol with Bacillus megaterium in the absence of a bacterial symbiont.

7. The method of claim 6, wherein contacting the polyvinyl alcohol with Bacillus megaterium comprises contacting the polyvinyl alcohol with a composition comprising a Bacillus megaterium strain having the deposit accession number B-68327 and a carrier, diluent, surfactant, or adjuvant.

8. The method of claim 6, wherein the bacterial symbiont is PN19.

9. A method of removing a sacrificial polymer from graphene comprising providing a sacrificial polymer adhered to graphene, and contacting the sacrificial polymer with a Bacillus megaterium strain, wherein the Bacillus megaterium strain degrades the sacrificial polymer.

10. The method of claim 9, wherein the sacrificial polymer is polyvinyl alcohol, polymethyl methacrylate, polydimethylsiloxane, or polycarbonate, or an α-angelica lactone polymer having the structure: 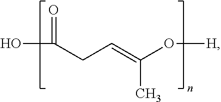
wherein n is a positive integer ranging from 1-100,000.

11. The method of claim 9, wherein the sacrificial polymer is a film.

12. The method of claim 9, wherein the graphene was grown by chemical vapor deposition on a formation substrate prior to adherence to the polyvinyl alcohol.

13. The method of claim 12, wherein the formation substrate is a transition metal.

14. The method of claim 12, wherein the formation substrate is copper foil.

15. The method of claim 9, wherein the graphene is adhered to a target substrate.

16. The method of claim 15, wherein the target substrate is an insulating material.

17. The method of claim 15, wherein the target substrate is SiO₂/Si or glass.

18. The method of claim 9, wherein the Bacillus megaterium strain degrades over 90% of the sacrificial polymer from the graphene.

19. The method of claim 9, wherein the Bacillus megaterium strain is averse to graphene.

20. The method of claim 9, wherein organic solvents are not used to remove the sacrificial polymer.

21. The method of claim 9, wherein the sacrificial polymer is polyvinyl alcohol and the Bacillus megaterium strain is the isolated Bacillus megaterium strain having the deposit accession number B-68327, and wherein contacting the sacrificial polymer with the Bacillus megaterium strain occurs in the absence of a bacterial symbiont.

22. The method of claim 21, wherein the isolated Bacillus megaterium strain having the deposit accession number B-68327 degrades over 90% of the polyvinyl alcohol from the graphene.

23. The method of claim 9, wherein the sacrificial polymer is an α-angelica lactone polymer having the structure: 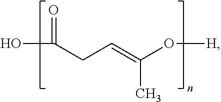
wherein n is a positive integer ranging from 1-100,000; and wherein, the isolated Bacillus megaterium strain has the deposit accession number B-68328.

24. The method of claim 9, wherein the isolated Bacillus megaterium strain does not contaminate the graphene.

25. The method of claim 9, wherein the graphene has improved optical and electronic properties as compared to graphene resulting from polyvinyl alcohol removal comprising a solvent treatment.

26. The method of claim 9, wherein contacting the sacrificial polymer with the Bacillus megaterium strain comprises contacting the sacrificial polymer with the composition of claim 4.

27. Graphene produced by the method of claim 9.

28. A method of producing a Bacillus megaterium strain that degrades an α-angelica lactone polymer comprising:
culturing Bacillus megaterium in a medium comprising an α-angelica lactone polymer having the structure: 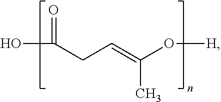
wherein n is a positive integer ranging from 1-100,000.

29. A method for degrading an α-angelica lactone polymer comprising: contacting the α-angelica lactone polymer with Bacillus megaterium.

30. The method of claim 29, wherein contacting the α-angelica lactone polymer with Bacillus megaterium comprises contacting the α-angelica lactone polymer with a composition comprising a Bacillus megaterium strain having the deposit accession number B-68328 and a carrier, diluent, surfactant, or adjuvant.
